(19) United States
(12) Patent Application Publication
(10) Pub. No.: US 2002/0091441 A1
(43) Pub. Date: Jul. 11, 2002

(54) Focused beam cutting of materials

(76) Inventor: Donald S. Guzik, Ramsey, MN (US)

Correspondence Address: Peter S. Dardi, Patterson, Thuente, Skaar & Christensen, P.A., 4800 IDS Center, 80 South 8th Street, Minneapolis, MN, 55402-2100, US

(21) Appl. No.: 09/755,424

(22) Filed: Jan. 5, 2001

(51) Int. Cl. A61F 2/24; G06F 19/00

(52) U.S. Cl. 623/2.13; 700/166; 623/901

(57) ABSTRACT

Methods are disclosed for producing a prosthesis by at least partially cutting a material segment with a beam. The cutting is preferably controlled by a process control unit to cut the material segment along a pattern to generate a target image and size. In some embodiments, the material segment includes a tissue segment separated from an organism. An apparatus for cutting tissue comprises a tissue segment, a support platform, a beam generator and a process control unit. The beam generator is oriented to direct a beam at the tissue segment supported by the support platform. The process control unit is operably connected to the support platform. The improved cutting approaches can be used in the formation of heart valve prostheses.

### BACKGROUND OF THE INVENTION

The invention relates to the use of a focused beam for the cutting of materials, such as tissue. In particular, the invention relates to precision cutting of tissue for incorporation into prostheses using a focused beam.

Various medical articles have been designed particularly for contact with a patient's body fluids. This contact can be sufficiently long such that surface interactions between the medical article and the patient's blood and/or tissue become significant. Components of these medical devices can be formed from tissue. Relevant medical articles include, for example, prostheses.

Prostheses, i.e., prosthetic devices, are used to repair or replace damaged or diseased organs, tissues and other structures in humans and animals. Prostheses must be generally biocompatible since they are typically implanted for extended periods of time. Prostheses can be constructed from natural materials, such as tissue, synthetic materials or a combination thereof.

Bioprosthetic heart valves from natural materials were introduced in the early 1960's. Bioprosthetic heart valves typically are derived from pig aortic valves or are manufactured from other biological materials such as bovine pericardium. Xenograft heart valves, i.e., heart valves originating from a donor of a species different from the species of the recipient, are typically fixed with glutaraldehyde or other crosslinking agent prior to implantation to reduce the possibility of immunological rejection. Glutaraldehyde and other fixatives generally react to form covalent bonds with free functional groups in proteins, thereby chemically crosslinking nearby proteins.

Although mechanical heart valves have the advantage of proven durability through decades of use, they are associated with a potential for blood clotting on or around the prosthetic valve. Blood clotting can lead to acute or subacute closure of the valve or associated blood vessel. For this reason, patients with implanted mechanical heart valves remain on anticoagulants after implantation of the valve. Anticoagulants impart a potential risk of bleeding complications and cannot be taken safely by certain individuals.

In contrast, tissue-derived prosthetic heart valves generally have blood flow characteristics and surface properties that provide a high degree of thromboresistance without the need for anticoagulant therapy. Therefore, thrombosis or thromboembolism and bleeding complications are less likely to occur than with mechanical heart valves. Prosthetic tissue heart valves are limited by a tendency to fail beginning about seven to ten years following implantation. Valve degeneration is particularly rapid in young patients and during pregnancy. However, different treatments have been developed and are continuing to be developed to reduce tissue degeneration following implantation of prostheses. As improvements in tissue treatments improve the durability of implanted tissue products, tissue based prostheses and the like will likely gain preference. Thus, bioprosthetic heart valves serve as an alternative to mechanical heart valves for heart valve replacement in certain individuals.

### SUMMARY OF THE INVENTION

In a first aspect, the invention pertains to a method for producing a prosthesis. The method includes at least partially cutting a material segment with a beam in which the cutting is controlled by a process control unit to cut the material to generate a target shape. The method is particularly suitable for the cutting of tissue segments and polymer sheets for producing prosthesis components.

In a further aspect, the invention pertains to an apparatus for cutting a tissue segment. The apparatus includes a tissue segment, a support platform, a beam generator, a motor and a process control unit. The support platform supports the tissue segment. The beam generator is oriented to direct a beam at the tissue segment. In addition, the process control unit controls the relative position of the support platform and the beam. The apparatus can further include a motor that changes the relative position of the support platform and the beam. In some embodiments, the process control unit can include a digital processor that is operably connected to the motor wherein the digital processor controls the motor based on a target pattern.

In another aspect, the invention pertains to a heart valve prosthesis comprising a tissue segment separated from the host, the tissue having a cauterized edge.

In addition, the invention pertains to a method of cutting a tissue sheet to remove portions of the tissue sheet having different thicknesses. The method includes imaging the tissue sheet and cutting the tissue sheet. The imaging of the tissue sheet is performed on a smooth surface to evaluate the thickness of the tissue sheet at different points. The cutting of the tissue sheet removes portions of the tissue sheet with a thickness outside of a selected range.

### BRIEF DESCRIPTION OF THE DRAWINGS

FIG. 1 is a perspective view of an aortic heart valve prosthesis formed from a valve explant with intact leaflets.

FIG. 2 is a perspective view of a mitral heart valve explant.

FIG. 3 is a side view of an aortic heart valve prosthesis assembled from tissue components.

FIG. 4 is a side perspective view of a four leaflet mitral valve prosthesis formed from tissue components.

FIG. 5A is a schematic view of an automated focused beam cutting apparatus for precision cutting for forming medical devices.

FIG. 5B is a schematic top view of an embodiment of a support platform for use with the cutting apparatus of FIG. 5A.

FIG. 6 is a top plan view of an example of a leaflet template, used as a guide with the cutting apparatus.

FIG. 7 is a plan view of an aortic leaflet cut from an aortic valve explant.

FIG. 8 is a plan view of a tissue segment including three aortic leaflets sewn together.

FIG. 9 is a plan view of the tissue segment of FIG. 8 showing the side opposite the leaflets.

FIG. 10 is a plan view of the tissue segment of FIG. 8 following the cutting of a portion of aortic wall to form commissure supports.

FIG. 11 is a plan view of the tissue segment of FIG. 8 following the cutting of the aortic wall around the three leaflets.

FIG. 12 is a plan view of the tissue segment of FIG. 11 following a rounding of the commissure supports.

FIG. 13 is a plan view of the tissue segment of FIG. 12 sewn to a tissue sheet.

FIG. 14 is a side view of the tissue components of FIG. 13 following the joining of the opposite ends of the tissue segment to form a valve structure and a partial trimming of the tissue.

FIG. 15 is a side view of a first leaflet section for the four leaflet heart valve prosthesis of FIG. 4.

FIG. 16 is a side view of a second leaflet section for the four leaflet heart valve prosthesis of FIG. 4.

FIG. 17 is a side view of a third leaflet section for the four leaflet heart valve prosthesis of FIG. 4.

FIG. 18 is a top view of a fourth leaflet section for the four leaflet heart valve prosthesis of FIG. 4.

FIG. 19 is a side perspective view of a cylindrical mandrel suitable for supporting a tissue segment.

### DETAILED DESCRIPTION OF THE ILLUSTRATIVE EMBODIMENTS

Precision cutting of tissue or other materials using a focused beam, e.g., radiation or water, for the formation of medical devices allows for the efficient production of tissue elements with greater reproducibility and uniformity. In preferred embodiments, the cutting is performed with an automated system, although manual beam cutting can be advantageously performed also. In an automated cutting process, the cutting process can be controlled to follow a stored pattern/image or a physical template to guide the reproducible cutting of the material. In alternative embodiments, the automated cutting process can rely on a digital image to direct the cutting based on differences between the digital image and a digital target image of desired configuration. The resulting cut tissue element can be produced with greater accuracy and consistency while reducing waste due to human error. While preferred embodiments involve the cutting of tissue segments, focused beam cutting can be advantageously applied to the cutting of other material segments, in particular polymers, for the fabrication of medical devices.

In general, relevant medical devices are prostheses or components of prostheses that are formed to mimic a corresponding structure within the human body. The prostheses can also be used to replace or repair the corresponding native structure. The medical devices generally are therefore suitable for long term implantation within a recipient patient. Generally, the patient is an animal, preferably a mammal, such as a human.

Prior to the cutting of a tissue segment, the tissue can be harvested either from the patient recipient, i.e., autograft tissue, from an individual of the same species as the patient, i.e., homograft tissue, or from an individual of a different species, i.e., xenograft tissue. The tissue can be subjected to additional processing, such as treatment with a sterilizing agent and/or a crosslinking agent, prior to cutting and/or after cutting. In preferred embodiments, the tissue is maintained in a hydrated form from harvesting through processing, although dehydrated and/or frozen tissue also can be subject to the precision cutting process.

Some devices, such as pericardial patches and dermal grafts for wound healing, are straightforward in structure. For example, pericardial patches can be generally rectangular sections of tissue. Even for these less complex structures, there can be advantages in decreasing the need for human contact by processing the tissue using focused beam cutting. The cutting process can also be used to cut the tissue to a desired thickness.

Furthermore, a variety of tissue based prostheses have complex structures that can be produced with greater efficiency and uniformity due to the precision gained using focused beam cutting. The processing of tissue based heart valve prostheses are of particular interest. Suitable heart valve prostheses include constructed valves in which cut tissue components are assembled, and intact tissue valves with leaflets that are suitably trimmed by precision focused beam cutting and assembled with implantation components to complete the prosthesis. For these more complex structures, the precision focused beam cutting approaches provide particular advantages.

For the cutting of both less-complex structures and complex structures with well-defined geometries, the cutting tool can be programmed to follow a preselected template, which can be an image or virtual template stored in digital form or a physical template. The virtual template or the physical template form a two or three dimensional pattern outlining the structure of the target tissue element. Cutting based on a virtual template or a physical template is particularly suitable for cutting tissue elements from a sheet of tissue. Template based cutting is also suitable for cutting non-planar components with well-defined geometries that can be aligned according to a preselected pattern.

As long as the initial tissue segment is appropriately placed within the cutting apparatus, the automated cutting procedure can properly cut the tissue along the virtual template. With a physical template, the cutting can be automated so that the beam is directed to follow the pattern of the template, or the physical template can physically block the beam such that only material not blocked by the template is cut as the beam is moved across the material. Alternatively, an operator can manually control a beam on a motorized stand to cut along a pattern dictated by the template.

In other embodiments, the cutting process uses a digital image, preferably a three dimensional image. Suitable imaging approaches include, for example, video imaging, computerized 3D enhancement and scanning phase measurements. These approaches are based on the use of light to image the object. To perform the imaging, the object can be illuminated for imaging with back lighting that is directed through the object and/or through a transparent support surface, or light reflected from the object can be measured. Measurements of images or reference points from different directions can be used to construct a 3-dimensional image. While preferred approaches are based on non-contact imaging, a probe could be used to form a contact based image.

The digital image of the actual tissue element is first aligned to match corresponding structure in a target image, corresponding to a desired shape. Once the digital image is aligned, any difference can be evaluated between the digital image of an object and a stored image of a desired shape. The stored image of a desired shape can be obtained from the digital image of a previously processed object with the desired characteristics or by fabricating a desired hypothetical shape, for example, using design software.

The difference between the digital image of the actual object and the digital image of the desired target article represents the material to be removed during the cutting process. Thus, this digital difference information can be used to direct the cutting operation by cutting along the contours dividing the two images. Using the image, the cutting can be controlled to avoid inadvertently cutting tissue that should remain intact. This approach is different from the template-based approach in that complex structures can be accounted for in the alignment process and interference with the beam by separate structural features can be avoided by examination of the image.

The cutting is performed by moving the tissue along a contour, within a plane or in three dimensions, relative to the focused beam that performs the cutting. This relative motion can be accomplished by moving the tissue segment and/or by moving the focused beam. Thus, the tissue can be mounted on a movable stage that guides the cutting process. Alternatively, the focused beam is scanned across the tissue. If the beam source, such as a laser, is too massive to move in a practical way, optical components or similar directing tools, such as conduits, mirrors, lenses and the like, can be moved instead to direct the focused beam.

Suitable focused beams includes, for example, a focused high intensity light beam or a fluid jet, in which the fluid can be a liquid, such as water, or a gas, such as air. The focused high intensity light beam can be a laser or a non-laser light source. The light source can have any optical frequencies, although preferred light sources include, for example, infrared lasers. In alternative embodiments, a focused fluid jet, such as a water jet, is used to perform the cutting.

The tissue can be hydrated or dehydrated during the cutting process. Similarly, the tissue can be frozen or not frozen during the cutting process. In preferred embodiments, the tissue is not frozen and is hydrated. Dehydration of the tissue can result in undesirable and irreversible structural changes. To prevent undesirable amounts of dehydration of the tissue during the cutting process, the tissue can be placed on the cutting surface in a moist form. Moisture on the cutting surface can be maintained, for example, by using a fluidized bed. Similarly, a sprayer can be used to spray liquid onto the tissue to maintain hydration. If the focused beam is appropriately configured, the tissue can be submerged in liquid.

Even if a layer of moisture is not maintained, if the cutting is performed quickly, even at ambient atmospheric conditions, the tissue will not significantly dehydrate during the cutting process. In addition, the ambient atmosphere can be kept at a high humidity to reduce the drying of the tissue. If desired, the cutting can be performed in a closed system, such as a glove box or climate controlled room, in an atmosphere with very high humidity levels to effectively eliminate any drying of the tissue.

Following cutting of the tissue, the cut tissue can be further processed to complete the prosthesis, if any additional processing is required. Chemical treatments, such as fixation, contact with biological agents, such as growth factors, and sterilization can be performed before and/or after tissue cutting. Additional structural processes can involve assembly of the prosthesis from one or more components. Simple structures, such as pericardial patches and dermal grafts, are complete structurally and are ready following the cutting for any additional nonstructural processing and then storage. Other prostheses, such as heart valve prostheses, may require at least the addition of a sewing cuff and/or other components to assist with implantation of the device. Heart valve prostheses may also require attachment of tissue elements, attachment to a stent and/or other structural supports to prepare the prostheses for implantation and proper function following implantation.

The manual cutting of tissue for medical use, such as forming prostheses, requires highly skilled technicians, although the process may be highly repetitive in some situations. Nevertheless, the importance of the cutting to form a reliable medical device necessitates the use of precision processing of the prostheses. Even with skilled technicians, errors made by manual cutting of tissue leads to considerable waste of components, such as porcine heart valves, that have significant preparation and handling expenses. Thus, the manual processing of tissue for the production of medical devices has a relatively high cost.

Cutting with focused beams provides for precision cutting of the tissue or polymers with reduced human interaction. If the process is automated, the cutting can be performed with increased uniformity, accuracy and with a decrease of waste. In addition, skilled workers can be freed for performing other tasks. Also, automated processing of tissue elements should increase throughput and yields. The consistency in tissue elements, such as leaflet size, will provide a more uniform device with more predictable performance.

Medical Devices

Preferred medical devices generally include a tissue material, although alternatively, the medical devices can be formed from other materials, in particular polymers, which may be combined with tissue. Generally, these medical devices are prostheses or components designed for implantation into or onto a patient for extended periods of time. Relevant prostheses include, for example, artificial hearts, artificial heart valves, annuloplasty rings, pericardial patches, vascular, coronary and structural stents, vascular grafts or conduits, permanently in-dwelling percutaneous devices, vascular shunts, dermal grafts for wound healing, and surgical patches.

Particularly preferred medical devices include tissue based heart valve prostheses. Tissue based heart valve prostheses can be stented, in which a stent serves as a frame for maintaining function of tissue based leaflets, or stentless, in which a tissue heart valve is implanted utilizing the recipient's native support structure, e.g., the aorta or papillary muscles attached to chordae, to maintain leaflet function. Heart valve prostheses can be constructed from segments of tissue that are cut to have a desired shape and assembled into a complete prosthesis. Alternatively, heart valve prostheses can be formed by trimming heart valve explants with intact leaflets.

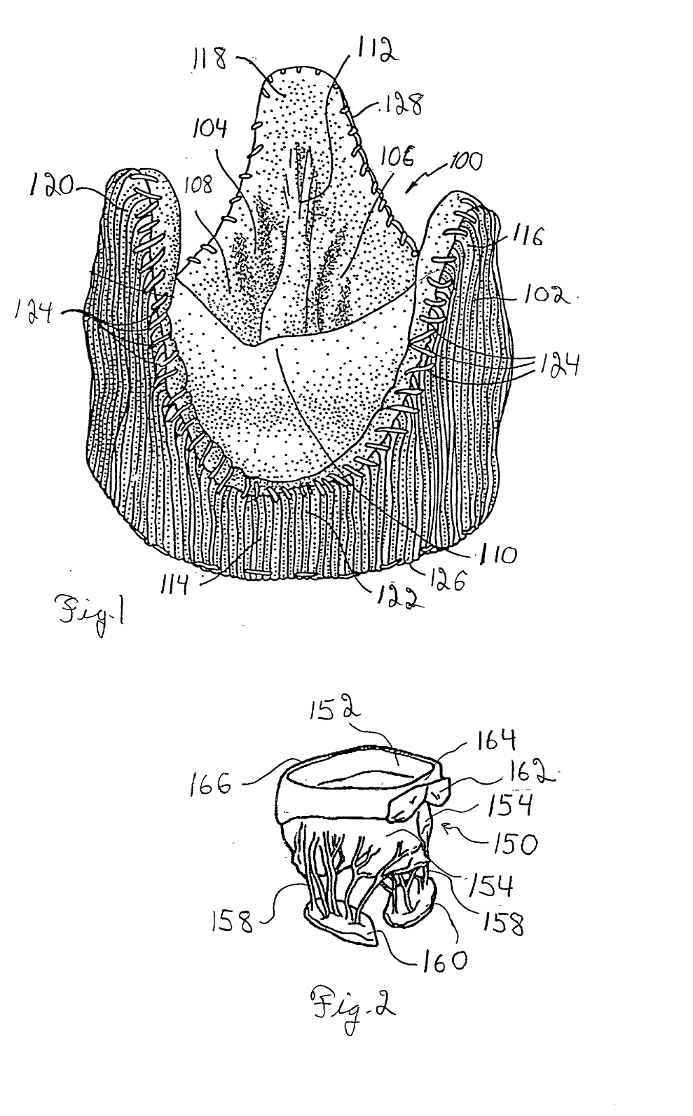

An aortic stentless heart valve prosthesis is shown in FIG. 1. Aortic valve 100 includes a cover 102 and a harvested heart valve explant 104. Heart valve explant 104 has three leaflets 106, 108, 110 meeting at commissures 112. Cover 102 has a generally annular base 114 and three commissure supports 116, 118, 120. Commissure supports 116, 118, 120 extend between scallops 122. Sutures 124 are located along inflow edge 126 and outflow edge 128 to secure cover 102 to heart valve explant 104. Heart valve explant 104 is appropriately cut prior to attachment to the cover 102.

An embodiment of a mitral stentless heart valve prosthesis is shown in FIG. 2. Harvested mitral heart valve explant 150 preferably includes an annulus 152, leaflets 154 which connect to annulus 152, chordae tendineae 158 which extend between leaflets 154 and portions of papillary muscle section 160. The leaflets 154, chordae 158 and papillary muscle section 160 form a complete subvalvular apparatus. Alternatively, papillary muscle section 160 can be replaced with a synthetic material, such as a polymer. Annulus 152 includes a remaining portion of porcine aortic valve 162 at the anterior mitral annulus 164. Posterior mitral annulus 166 is formed mostly with atrial muscle, while the region of anterior annulus 164 is supported by a fibrous skeleton, i.e., mitro-aortic tendinous tissue. As described further below, annulus 152 and papillary muscle sections 160 can be trimmed by the methods described herein.

A trileaflet, stentless, assembled aortic heart valve prosthesis is shown in FIG. 3. Aortic prosthesis 170 includes three leaflets 172, 174, 176 excised from a porcine aortic heart valve. Leaflets 172, 174, 176 are joined at commissures supports 178, 180, 182. Leaflets 172, 174, 176 are secured to a section of bovine pericardium 184 or other appropriate material with suture 186 or other fastener. Bovine pericardium 184 holds the valve structure together. As described further below, the leaflets are cut prior to attachment to the pericardial tissue. The pericardial tissue generally is also cut prior to attachment to the leaflets. Also, further cutting may take place following the assembly of the leaflets on the bovine pericardium. In alternative embodiments, the bovine pericardium is replaced with a synthetic material or other tissue material.

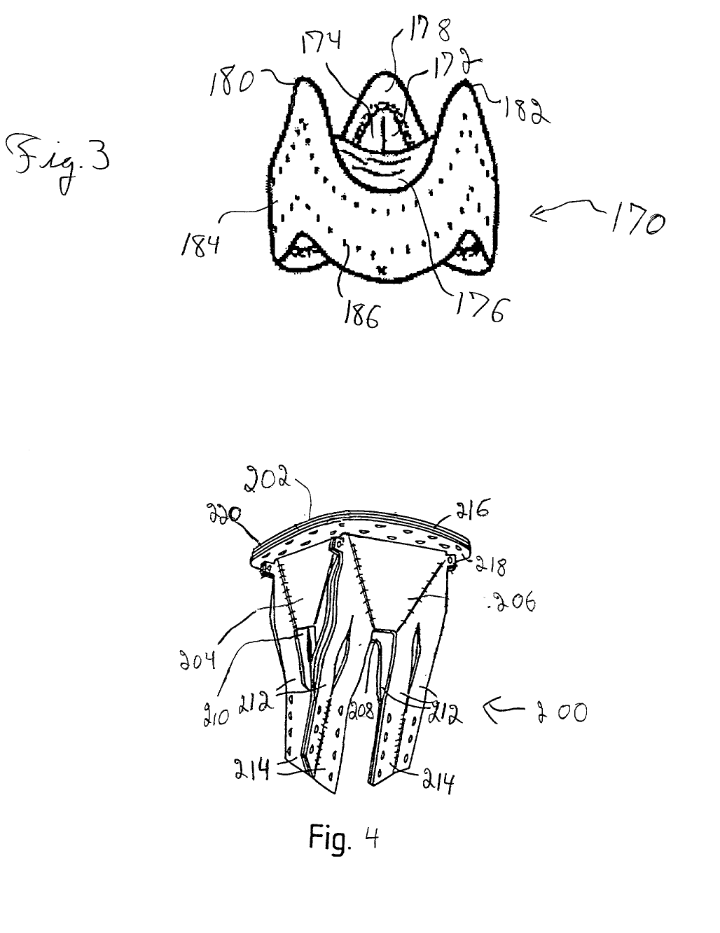

A stentless mitral valve prosthesis with four leaflets assembled from tissue elements is shown in FIG. 4. Heart valve 200 includes a sewing ring 202, and four leaflets 204, 206, 208, 210. Chordae 212 extend from edges of leaflets 204, 206, 208, 210. Chordae 212 can be formed from a single sheet of biocompatible material, such as tissue, as described further below. Chordae 212 connect with attachment sections 214 for attachment to the patient's papillary muscles. An edge 216 of the tissue forming leaflets 204, 206, 208, 210 is stitched between two portions 218, 220 of sewing ring 202 to secure the leaflets to the sewing ring. The components, including the leaflets and sewing ring, can be cut using a focused beam, and preferably, a physical template or a template image.

Tissue

Appropriate bioprosthetic tissue materials can be formed from natural materials, synthetic tissue matrices and combinations thereof. Synthetic tissue matrices can be formed from extracellular matrix proteins that are crosslinked to form a tissue matrix or from synthetic materials, such as polymers, that have or have had viable cells associated with the matrix. Thus, tissue materials have viable cells or structures formed from viable cells that are no longer present. Extracellular matrix proteins are commercially available.

Natural, i.e. biological, tissue material for use in the invention includes relatively intact tissue as well as decellularized tissue. These natural tissues may be obtained from, for example, native heart valves, portions of native heart valves such as roots, walls and leaflets, pericardial tissues such as pericardial patches, amniotic sacs, connective tissues, bypass grafts, tendons, ligaments, skin patches, blood vessels, cartilage, dura mater, skin, bone, fascia, submucosa, umbilical tissues, and the like.

Natural tissues are derived from a particular animal species, typically mammalian, such as human, bovine, porcine, canine, seal or kangaroo. These tissues may include a whole organ, a portion of an organ or structural tissue components. Suitable tissues include xenografts, homografts and autografts. These natural tissues generally include collagen-containing material. Natural tissue is typically, but not necessarily, soft tissue. Tissue materials are particularly useful for the formation of tissue heart valve prostheses. The tissue can be decellularized. Decellularization approaches are described, for example, U.S. Pat. No. 5,855,620 to Bishopric et al., entitled “Matrix Substrate for a Viable Body Tissue-Derived Prosthesis and Method for Making the Same,” incorporated herein by reference.

Tissues can be fixed by crosslinking. Fixation provides mechanical stabilization, for example, by preventing enzymatic degradation of the tissue. Glutaraldehyde, formaldehyde or a combination thereof is typically used for fixation, but other fixatives can be used, such as epoxides, diimides and other difunctional aldehydes. In particular, aldehyde functional groups are highly reactive with amine groups in proteins, such as collagen.

Besides crosslinking, the tissue can be treated with other compounds to modify the tissue properties. In preferred embodiments, the tissue is treated with calcification reducing compounds. For glutaraldehyde crosslinked tissue, preferred anticalcificaton agents include, for example, multivalent metal cations, such as Al⁺³. The use of polyvalent metal cations is described, for example, in U.S. Pat. No. 5,368,608 to Levy et al., entitled “Calcification-Resistant Materials and Methods of Making Same Through use of Multivalent Cations,” incorporated herein by reference. The delivery of anti-calcification agents using microscopic storage structures is described in copending and commonly assigned U.S. patent application Ser. No. 08/931,930 to Schroeder et al., entitled “Calcification Resistant Biomaterials,” incorporated herein by reference. The tissues can be treated with other agents to impart desirable properties, such as growth factors and the like.

The tissue material can form the entire medical device or it can form portions of the medical device. Similarly, different portions of tissue material can be combined to form the medical device. In particular, some heart valve prostheses are formed from intact heart valve explants harvested from an animal. The explants can be subsequently treated, for example with a crosslinking agent, and modified with appropriate natural or synthetic materials, such as a sewing cuff, to aid in attachment. Such embodiments are shown, for example, in FIGS. 1 and 2. In other preferred embodiments, segments of tissue are cut to a desired shape and assembled to form the desired prosthetic valve, with or without a rigid stent. Relevant embodiments with assembled tissue elements are shown, for example, in FIGS. 3 and 4.

Cutting Apparatus

A preferred cutting apparatus includes the capability for automated control of the cutting process, although some alternative embodiments provide for manual control of a motorized cutting apparatus. To perform the automated cutting, the tissue or other material, such as a polymer material, is mounted on a stage, a mandrel or similar support platform. The cutting beam can be moved across the tissue to perform the desired cutting by moving the support platform, moving the beam source or moving focusing optics to direct the beam at a selected location on the tissue. The cutting can be programmed to follow a stored pattern or physical template that results in a section of cut tissue with a desired shape. Alternatively, an imaging device can be used to generate a digital image of the tissue or a portion of the tissue that is to be cut. The digital image is compared with a desired target image such that the difference between the digital image and the target image represents the tissue to be removed by cutting.

In preferred embodiments, the tissue is kept moist during the cutting process. Thus, the tissue segment can be placed on a fluidized bed to keep the tissue moist, or a sprayer can be used to maintain tissue moisture. In other embodiments, the cutting apparatus can be placed in a controlled environment such as a glove box or the like. Within the glove box, the moisture level can be kept high to retard evaporation of moisture from the tissue. Thus, if the tissue is initially moist, the tissue will not significantly dry out during the cutting process. The humidity level can be controlled to approach 100 percent relative humidity. Similarly, the cutting apparatus can be operated in a room with a controlled environment with a high relative humidity level.

The tissue can also be submerged in an aqueous solution during the cutting operation. This is especially feasible for cutting along a template using a laser beam with a wavelength not significantly absorbed by water, such as visible light. The cutting may be complicated by diffraction of light at the interface between the liquid and air. However, the laser can be directly projected through a lens into the solution to reduce or eliminate complications due to refraction. A suitable apparatus to submerge a lens connected to a mirror tube to deliver a laser beam is described in U.S. Pat. No. 5,938,954 to Onuma et al., entitled “Submerged Laser Beam Irradiation Equipment,” incorporated herein by reference. The tissue and lens can be scanned along a template at a relatively slow rate to avoid forming waves in the liquid that could generate optical distortions.

While the discussion herein focusses on preferred embodiments involving tissue, the apparatus can also be used for the cutting of other materials for medical devices, in particular polymers. For embodiments involving the cutting of polymer elements of medical devices, generally moisture is not an issue. Appropriate laser wavelengths for cutting polymers are given below.

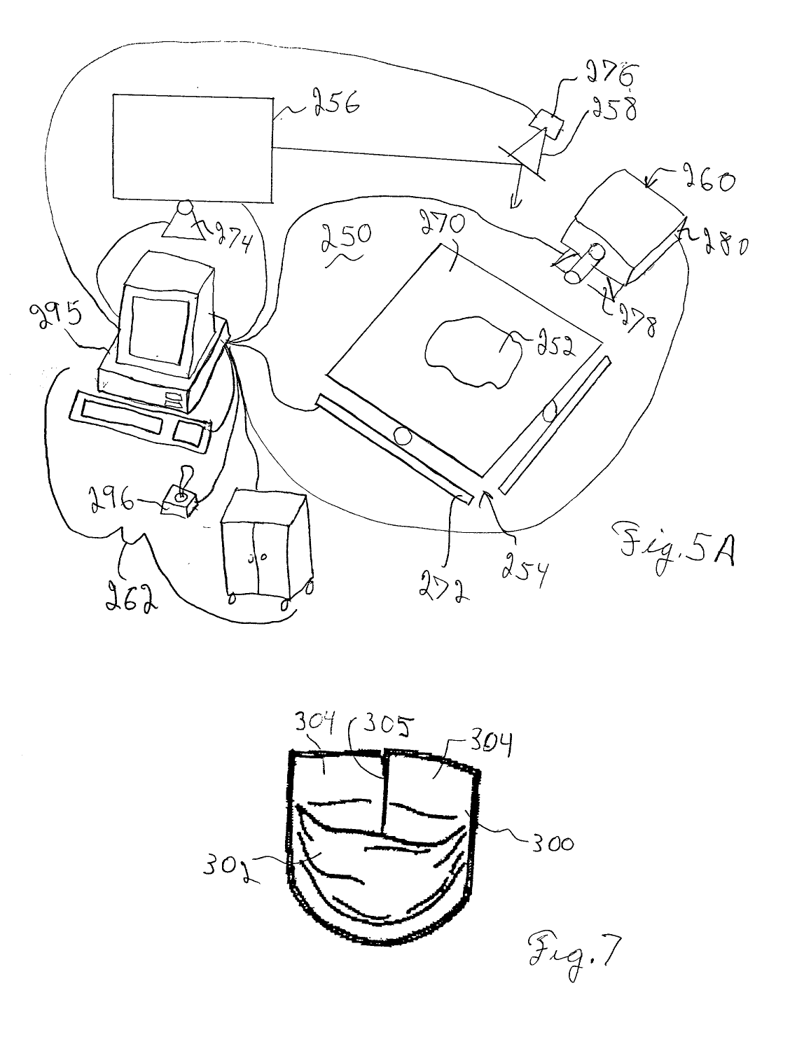

A suitable cutting apparatus is shown schematically in FIG. 5A. Cutting apparatus 250 can include, for example, a tissue segment 252, a support platform 254, a focused beam source 256, optics 258, an imager 260 and a process control unit 262, which generally includes a processor. The cutting apparatus can further include a physical template. Tissue segment 252 can be a valve explant from an animal source, a sheet of tissue or any other suitable piece of tissue. Tissue segment 252, for example, can be clipped to support platform 254, or retained to the support platform with various means, such as vacuum, or placed on a mandrel to hold tissue segment 252 in a desired orientation without damaging delicate portions of the tissue, or placed flat directly on support platform 254 without any fastening.

Support platform 254 includes a support surface or mount 270, which can be generally planar or curved, such as a mandrel. As noted above, support surface 270 can include clips, a holder or the like to properly support or retain tissue segment 252 during the cutting operation. If the beam is a light beam, platform 254 can include a transparent cover for containment of by-products, such as tissue, water, polymer and/or gases. In some embodiments, support platform 254 includes a motorized stand 272 that positions the platform at desired coordinates. For example, motorized stand 272 can include tracks that move the support surface 270 along three axes, i.e., forward and back, sideways, and up and down. Preferably, the motorized stand moves in small increments at a selectable speed. For example, repeatability can be obtained with the use of a stepper motor controlled by process control unit 262. Position and/or speed of the platform generally is controlled by process control unit 262. Alternatively or in addition, support platform 254 can include a manually manipulated stand and/or a manual override of the motorized stand. Manual control can involve the turning of knob, movement of levers, and the like, which may or may not be calibrated numerically.

One embodiment of a support platform is shown in FIG. 5B. Support platform 282 includes a fluidized bed 284 with suitable particles that become fluidized upon flowing a liquid through the bed. Liquid from a liquid source flows through a conduit 286 to a manifold 288 from which the liquid enters the fluidized bed. The liquid exits the bed at drain 290. The liquid can be recirculated. Tissue 252 is placed on a vacuum fixture 292 with a series of holes or inlets below the specimen to hold the tissue without damaging the tissue. Vacuum fixture is connected to a vacuum pump 294. Flow through the vacuum fixture can be designed to be low enough not to interfere significantly with the flow across the fluidized bed to drain 290. Any liquid flowing into the vacuum fixture can be recycled into the liquid source. Liquid flow within the fluidized bed and/or the vacuum fixture can be controlled by the process control unit. Alternatively, a sprayer can either pass periodically over the tissue to maintain the moisture level or provide continuous spray from a fixed position.

Referring to FIG. 5A, beam source 256 can be a laser, an intense light source, such as a flash lamp, a fluid jet, or other high energy focused beam. Beam source can include a support 274 to move and/or reorient the beam source to direct the beam to a desired direction. A wide range of light wavelengths can be used to perform the cutting. In addition, the light emissions from the source can pass through frequency shifting optics, such as a Raman cell, or non-linear optical elements to multiply the frequency, so as to obtain more desirable light frequencies.

With respect to suitable light wavelengths, infrared light sources with wavelengths greater than about 1 micron can be used. Suitable infrared laser include CO₂ lasers with emissions at about 10.6 microns and Neodymium YAG (Nd-YAG) lasers with emissions at about 1.3 microns and about 1.06 microns. The 1.3 micron emission has been found to be more suitable for cutting most tissue during surgery, although the 1.06 micron infrared light has been found to be particularly effective at cauterizing tissue. A laser beam will generally cauterize the edge being cut to varying degrees while not damaging the crosslinking or the surrounding tissue as well as correspondingly melting the edge of a polymer. Also, a hydrogen filled Raman cell at 5 to 30 atmospheres can be used to shift a portion of the about 1.06 micron wavelength light to about 1.9 microns for improved cutting. The use of a Raman shifting cell is described further in U.S. Pat. No. 5,180,378 to Kung et al., entitled “Laser Surgery System,” incorporated herein by reference.

Infrared light has the disadvantage that it cannot be seen. While this may not be important for the operation of the automated cutting apparatus, observing the light can be useful for initially aligning the system. A visible laser can be directed along the same light path as the infrared laser light for visible alignment of the beam path.

Visible light from about 350 nm to about 800 nm can also be used to perform the cutting. Titanium-sapphire lasers have emissions at about 760 nm, which are suitable for cutting soft tissue. Helium-neon lasers have an output at about 632 nm. Suitable diode lasers have output in the range from about 400 nm to about 1.6 microns covering the visible and near infrared range. Similarly, dye lasers can be used to produce intense visible light at selected wavelengths.

In addition, near ultraviolet radiation with wavelengths from about 250 nm to about 350 nm is also effective to cut tissue. Visible light with a wavelength from about 500 nm to about 700 nm can be frequency shifted using standard frequency doublers, such as nonlinear optical elements, to produce the desired ultraviolet radiation. Similarly, Nd-YAG lasers can be equipped with a frequency quadrupler to produce near UV radiation. Alternatively, a xenon fluoride laser can be used to directly produce suitable ultraviolet light. The use of ultraviolet light for performing surgery is described further in U.S. Pat. No. 4,791,927 to Menger, entitled “Dual-Wavelength Laser Scalpel,” incorporated herein by reference. For the cutting of polymers segments separately or along with tissue, visible and ultraviolet light is preferred since infrared light may melt or deform some polymers.

In preferred embodiments, a laser light source has a power output of at least about 1 milliwatt and more preferably from 50 milliwatts to about 500 watts and even more preferably from about 150 milliwatts to about 100 watts. Suitable power levels may depend on the thickness and nature of the tissue. Either continuous light emissions or pulsed light emissions can be used. For example, suitable pulsed lasers can pulse from about 1 nanosecond to several milliseconds. Short pulses on the order of 10 ns can have repetition rates on the order of about 10 hertz to about 1000 hertz. Pulse energies preferably range from about 0.01 joules to about 10 joules. Suitable lasers are available from a wide range of commercial suppliers for adaptation into the present apparatus.

In addition, a high pressure fluid can be used for tissue cutting. Preferred fluids include, for example, water or aqueous solutions, such as buffers, and air or other gases. Fluid jet cutters include a pump to force the fluid under very high pressure through a nozzle. In preferred embodiments, the fluid is forced through one or more very fine orifices to create a hair thin fluid jet for precision cutting. The fluid jet may operate under a pulsed operation.

The pump is used to deliver the fluid under high pressure to a nozzle. For pulsed flow, a diaphragm or other pump can be used. A delivery tube connects the nozzle with the pump. Depending on the nature of the tissue, the pressure leading to the nozzle generally ranges from about 1,000 psi (pounds per square inch) to about 50,000 psi and preferably from about 5,000 psi to about 30,000 psi. In pulsed operation, a repetition rate, for example, from about one pulse per second to about 100,000 pulses per second can be used. Similar fluid jet systems have been contemplated for surgical applications, as described, for example, in U.S. Pat. No. 5,871,462 to Yoder et al., entitled “Method For Using Fluid Jet Cutting System,” incorporated herein by reference.

In some embodiments, optical elements 258 are optional. In other embodiments, optics 258 can include various components for directing and/or focusing the cutting beam. Optics 258 can be used for embodiments with light radiation or fluid beam cutting. In particular, optics 258 can be beam directing optics, such as mirrors, fiber optic cables, prisms, conduits and the like, and focusing/flow control type optics, such as flow jets, nozzles, filters and lenses. Optics 258 can include an optional actuator 276 to position optics 258 at a desired location and/or orientation.

In preferred embodiments, actuator 276 is connected to process control unit 262 such that process control unit 262 can control the movement of actuator 276. Actuator 276 optionally includes a motor and is connected to the optical component in a suitable manner for the particular component, such as using a commercial lens holder to mount a lens. As a further example, actuator 276 can move a fiber optic cable and lens over the two dimensions of a hemisphere to position the cutting beam at a desired orientation.

Imager 260 is an optional component that is used to form one or more digital images of the tissue to guide the cutting process. Imager 260 generally includes a support 278 that positions one or more imaging devices 280 at one or more desired locations to perform the imaging. Imaging device 280 can be based on a variety of principles. Imaging devices 280 are discussed in detail in the following section. In preferred embodiments, imager 260 is connected to process control unit 262.

Process control unit 262 includes various circuit boards, connectors, computers, ports and the like to control the cutting process, and may have other elements, such as a cooling fan. Process control unit 262 may control the support platform, cutting beam, optical elements, hydration of tissue and other features. Process control unit 262 preferably includes one or more digital processors 295. In alternative embodiments, process control unit 262 can include a manual control unit 296 that can be used for manual control of the motorized motion of tissue 252 relative to the beam from optics 258 or beam source 256. Manual control (manual override) can be in addition to, or as an alternative, to control directly the cutting process under the control of the processor 295. The manual control can be directed through processor 295 to effectuate the manual commands.

Any suitable processor 295 can be used, such as a Pentium III® chip. In particular, processor 295 can be a dedicated processor designed for the operation of the cutting apparatus. Alternatively, processor 295 can be a conventional personal computer or work station operating on MacIntosh®, Windows®, Unix@, Linux® or other convenient operating system. Suitable software for operating the cutting operation includes, for example, LabVIEW® software available from National Instruments Corp., Austin, Tex. Specifically, LabVIEW® software can be used as a platform to program and coordinate the entire cutting operation, including any imaging software, and control software operating the stage. Suitable software is also available from View Engineering, Anaheim, Calif.

Imaging

To facilitate automated cutting of a portion of tissue, a digital image can be formed of the tissue segment prior to and/or during and/or after the cutting process. Suitable digital images can be formed from contact or non-contact imaging systems. Contact imagers use one or more contact probes that gently contact points on the surface of the object to ascertain the surface geometry. In preferred embodiments, a non-contact optical imager is used. A digital image can be formed using several different optical technologies, such as forming a video image, which can be evaluated using computerized 3D enhancement with available imaging software, or performing a scanning phase measurement.

A digital representation of the desired final structure, i.e., target structure, can be similarly generated and stored. The target structure can be generated using conventional drawing software or by taking digital images of desired structures formed using a suitable cutting technique. The difference between the digital image formed of the tissue being processed can be compared with the digital image of the target structure, i.e., the target image. The comparison can be completely automated, or a computational analysis, for example using CAD (Computer-Aided Design) software, can be displayed showing the differences, such that the operator can confirm, select or alter a potential cutting pattern. The comparison can provide the basis for further processing of the tissue.

To form a digital image of the tissue segment, a video imaging system can be used. Digital video cameras are readily available commercially. A ring illuminator can be provided around the lens of the video camera to uniformly illuminate the tissue segment. Suitable ring illuminators based on fiber optics are available from Edmund Scientific. Multiple views can be obtained by either moving the video camera to selected locations or by moving the tissue on the support platform to orient the tissue in desired orientations. Multiple images utilizing various reference points can be used to generate a three dimensional view of the object.

In alternative embodiments, a projected pattern of light is scanned across the object to be imaged at a relatively constant linear rate. For example, a grid of lines is projected across the surface of the object. The light reflected from the object is detected, for example, with a charge coupled device (CCD) array detector. The depth at a point is evaluated by the reading of the CCD element corresponding to the particular point on the object, as the light pattern is scanned across the tissue. Thus, a three dimensional image is formed. Generation of images using scanning phase measurements is described further in U.S. Pat. No. 5,646,733 to Bieman, entitled “Scanning Phase Measuring Method and System for an Object at a Vision Station,” incorporated herein by reference.

Various approaches have been developed for machine vision involving comparisons between images of an object and stored images to correlate the image with stored images. For example, a procedure to perform comparisons of images for quality control is described in U.S. Pat. No. 5,481,619 to Schwatz et al., entitled “Inspection Method Using Template Images, Unique Histogram Analysis, and Multiple Levels Correlated to Addresses of Template Images,” incorporated herein by reference. These approaches can be adapted directly to the cutting processes of interest. For instance, a tissue image can be compared to a “template” image when they are superimposed. Different images are created in the form of histograms. These could be used to direct a cutting tool to remove a portion of tissue to conform the image of the object with a target image.

More sophisticated aligning algorithms can be used also. For example, selected features can be used to align images of the tissue with the target image based on commercially available three dimensional display software. Once the images are aligned, the differences between the actual and target images can be evaluated. Alignment of images based on selected features is described in U.S. Pat. No. 5,875,004 to Yamane et al., entitled “Image Processing Inspection Apparatus,” incorporated herein by reference, and in U.S. Pat. No. 5,982,945 to Neff et al., entitled “Image Processing Method and Apparatus for Correlating a Test Image With a Template,” incorporated herein by reference.

Once images are aligned, the border or contour between the tissue image and the target image provides a guide for the cutting. By cutting along the border between the two images, the excess tissue is removed, and the tissue is made to conform to the target image. The actual image of the object can also be used to verify that no intact tissue is in the way of a desired cut. To avoid undesired cutting of tissue, a particular cutting angle can be changed to perform the desired cut. Using these techniques, variability due to the geometry of valves can be reduced or controlled to achieve desired configurations, or a harvested valve or portions of the valve (e.g., the leaflets) can be cut to a smaller desired size by removing excess portions of tissue.

Cutting Process

Various cutting operations can be accomplished using the apparatus and methods described herein for the preparation of medical devices and their components. The discussion below focuses on the cutting of tissue. However, the cutting of polymers and polymer/tissue combinations can generally be performed in a similar manner.

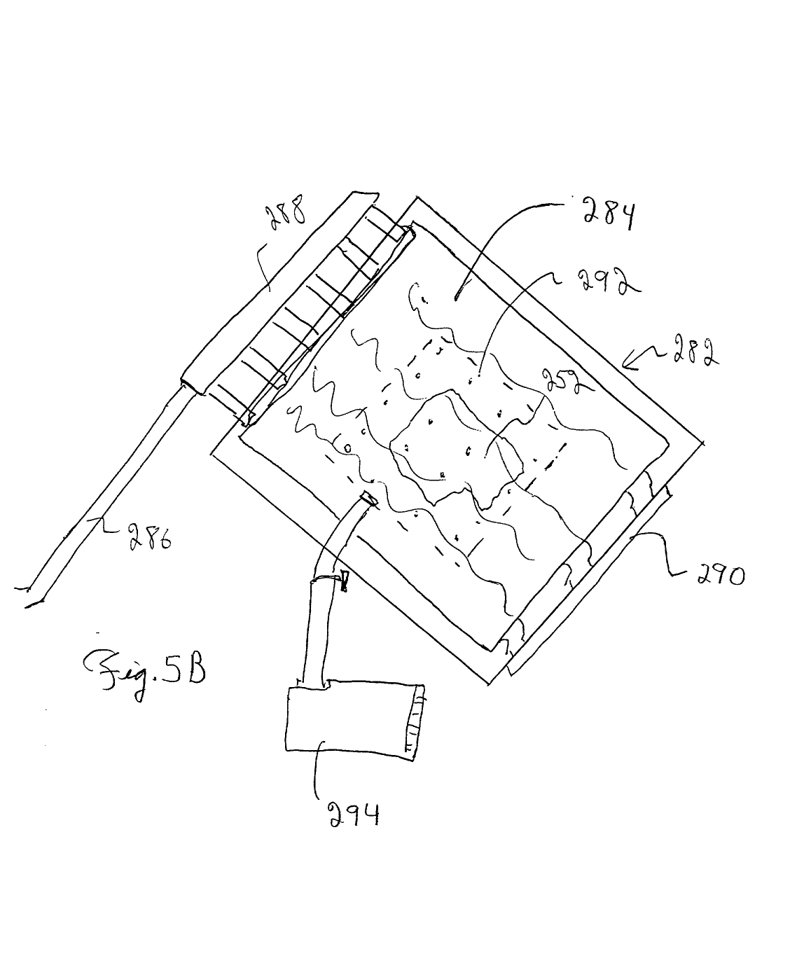

In one cutting approach, uniform tissue elements are formed by cutting a tissue segment along coordinates defined by a template. The template can be a physical template or a virtual template stored as a digital image in the process control unit. An example of a template for the cutting of leaflets is shown in FIG. 6, in which the lines in pattern 298 mark the cutting line for the focused beam to follow, for example, through the motion of a stylus or the like directing the beam. In other embodiments, the beam is scanned across the material, and the physical template blocks the beam to prevent the cutting of material under the template such that only exposed material is cut.

Alternatively, an image is generated of the tissue segment and cutting is performed based on differences between an image of the tissue segment and a target image. The cutting is programmed to remove tissue to conform the initial tissue segment image to the target image. In other alternative embodiments, imaging is used to remove unwanted structures without reference to a target image. For example, a sheet of tissue can be cut to remove portions that deviate from a desired range of thickness, as described further below.

While the discussion herein focuses on cutting, a focused beam can also be used to score or partially cut a material for use in a medical device. Scoring, for example, of tissue or a polymer material, can aid in the efficiency of fabrication of the medical device by making the components of the prosthesis more pliable and by reducing assembly time for fabrication of the prosthesis. Scoring can be performed by adjusting the beam energy and cutting speed such that the beam does not cut completely through the material. Generally, cutting herein refers to complete cutting through the material, although it will be recognized that in some embodiments, scoring may be sufficient.

Template based cutting is particularly useful for cutting planar sections of tissue for assembly into a prosthesis. Imaged based cutting is particularly useful for trimming valve explants to conform to desired parameters. While the cutting process is preferably automated and controlled by the PCU, the apparatus preferably includes a manual override, such that the operator can direct the cutting to include a variation in the standard procedure based on a particular tissue segment.

The tissue cutting can be performed advantageously using automated cutting methods at various stages of the tissue preparation process. For example, many of the tissue cutting steps can be performed under environmentally controlled conditions, possibly following one or more chemical treatments to the tissue. In addition, automated cutting procedures can also be advantageously performed at early stages of the procurement process, such as shortly after harvesting the tissue. More accurate trimming of crude versions of the valve following extraction from the host can reduce processing time and increase yield.

Template based cutting is useful for the production of many different types of heart valve prostheses. In some preferred embodiments, a sheet of tissue can be cut based on one or more templates to form individual leaflets or multiple leaflets. In other preferred embodiments, a template is used to form individual leaflets from valve explants.

For example, in one embodiment, a heart valve prosthesis can be formed from leaflets that are removed from an extracted valve and that then are assembled into a complete valve prosthesis. First, a porcine or other valve is obtained from a suitable source and stored under appropriate storage conditions until further processing is performed. The tissue generally is crosslinked prior to further manipulation of the tissue, although crosslinking can be performed after cutting the tissue. The initial valve can be oriented on a stage to allow for precision cutting of the valve with a focused beam of the cutting apparatus into three sections as shown in FIG. 7, although the number of sections can vary. Any leaflets that appear to be damaged or calcified can be discarded. This initial cutting and the remaining tissue cutting for this prosthesis can be made either with a template or with digital imaging.

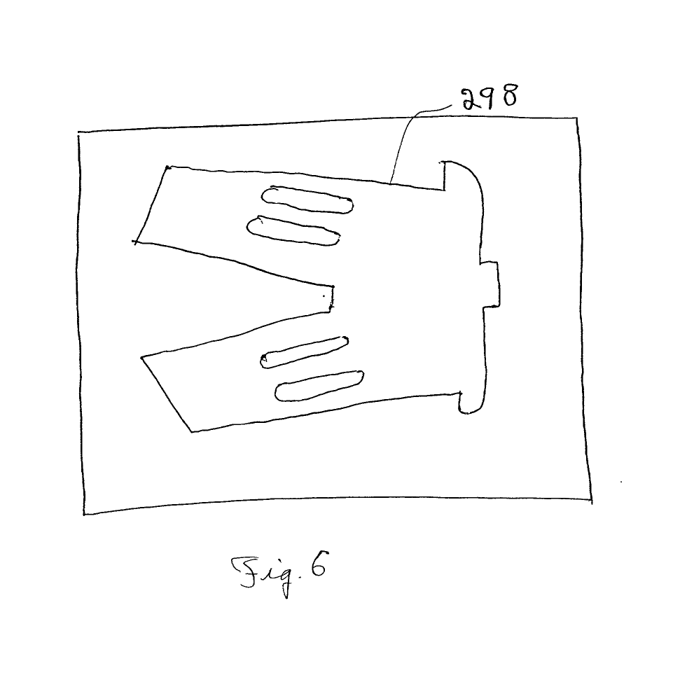

The resulting leaflet sections 300 include an uncut cusp or leaflet 302 and an aortic wall portion 304 that involves a cut 305 with a focused beam through the aortic wall to provide flexibility for the subsequent assembly. Cut 305 of aortic wall portion 304 is made while avoiding the cutting of leaflet 302. Three of the trimmed leaflet sections 300 that have been cut from one or more valves can be stitched/sutured, adhered or otherwise joined together to form a joined tissue segment 306 with three leaflets 302, as shown in FIGS. 8 and 9. In other embodiments, different numbers of leaflets are sutured together.

If desired, the leaflets 302 themselves can also be trimmed. For example, some leaflets have a thickening, called a muscle bar, toward the free edge of the leaflet. A leaflet can be cut with a focused beam generally parallel to the free edge using a focused beam cutting apparatus to reduce or eliminate the muscle bar. The resulting smaller leaflet can be used in the production of a smaller size valve. For example, an initial 33 mm leaflet can be cut to the size of a 26 mm leaflet while reducing or eliminating the muscle bar.

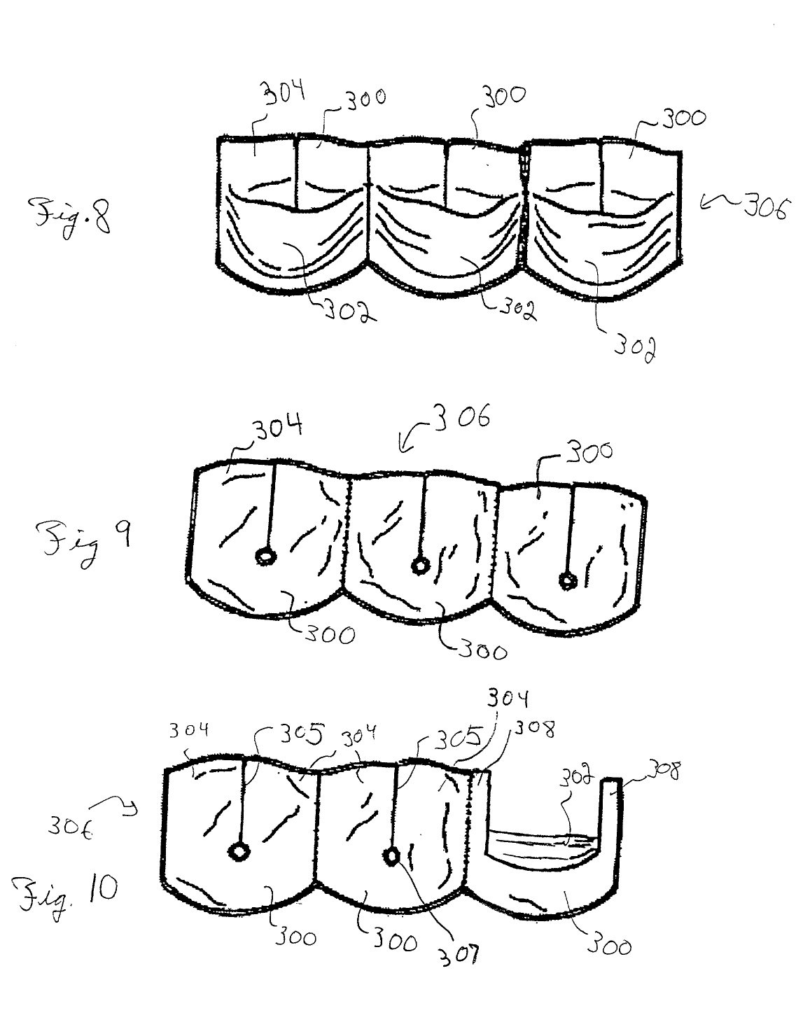

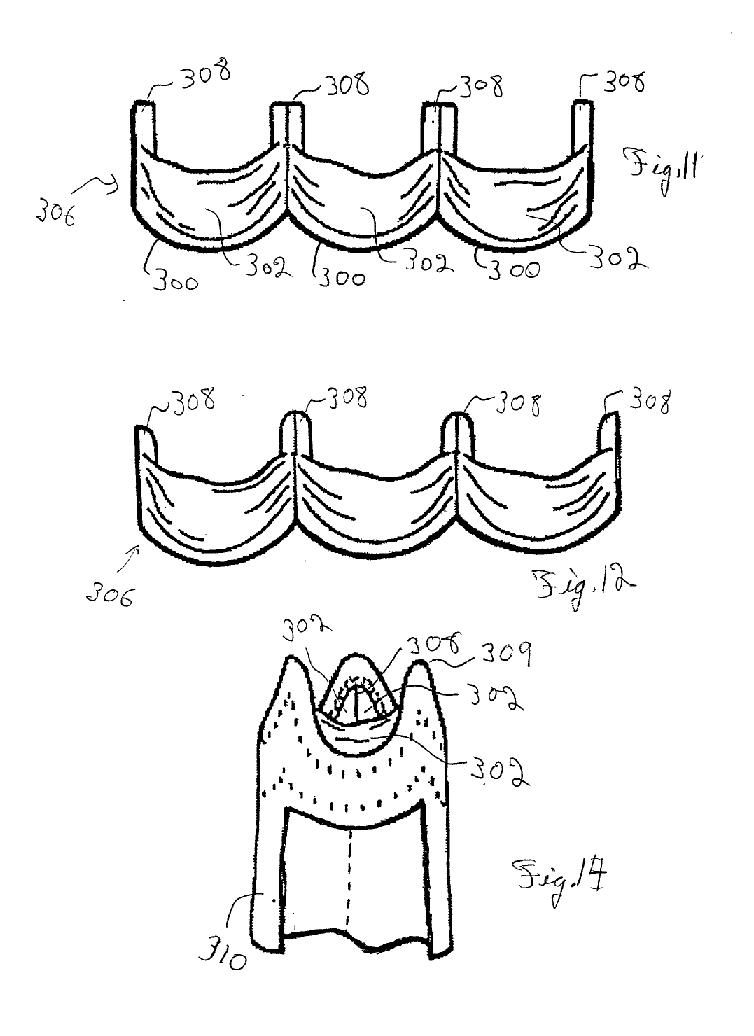

In one preferred embodiment, a portion of the aortic wall associated with each leaflet section 300 is then trimmed away to leave commissure supports 308 near the edge of each leaflet 302. Specifically, the aortic wall can be trimmed below the ostia 307 using a focused beam without cutting the leaflet 302, as shown in FIGS. 10 and 11. The tops of the commissure supports 308 can be cut to round the shape, as shown in FIG. 12.

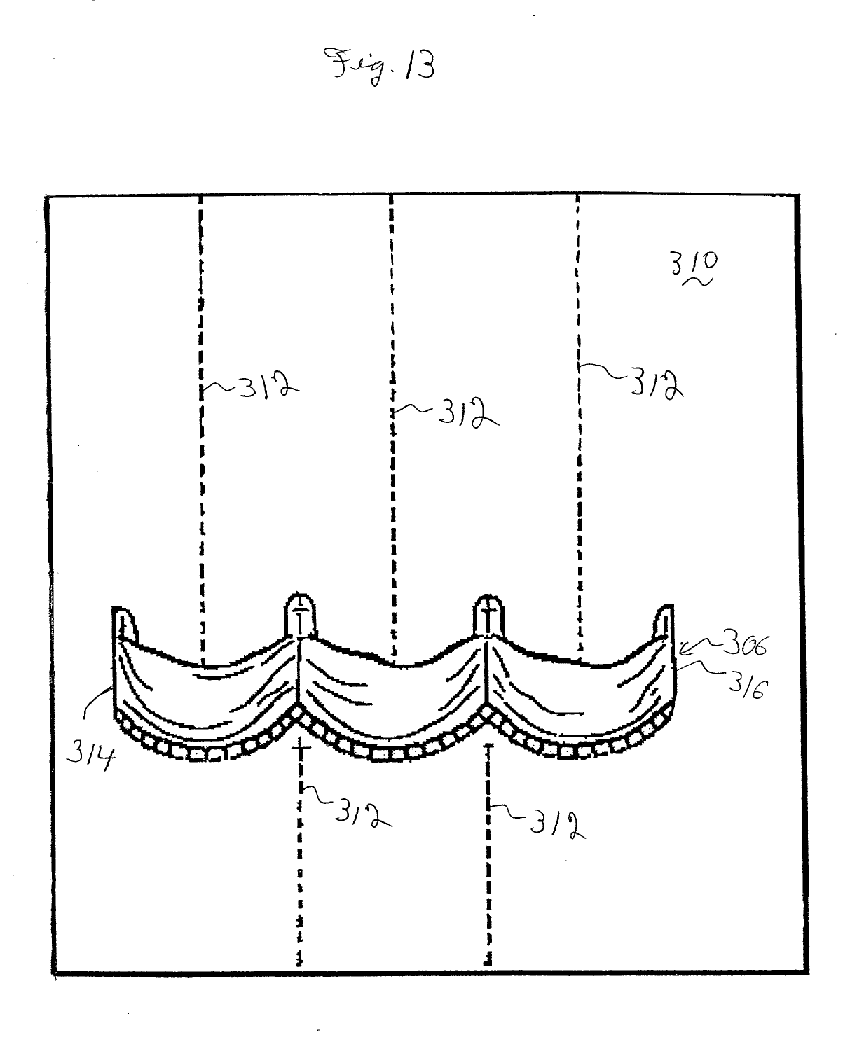

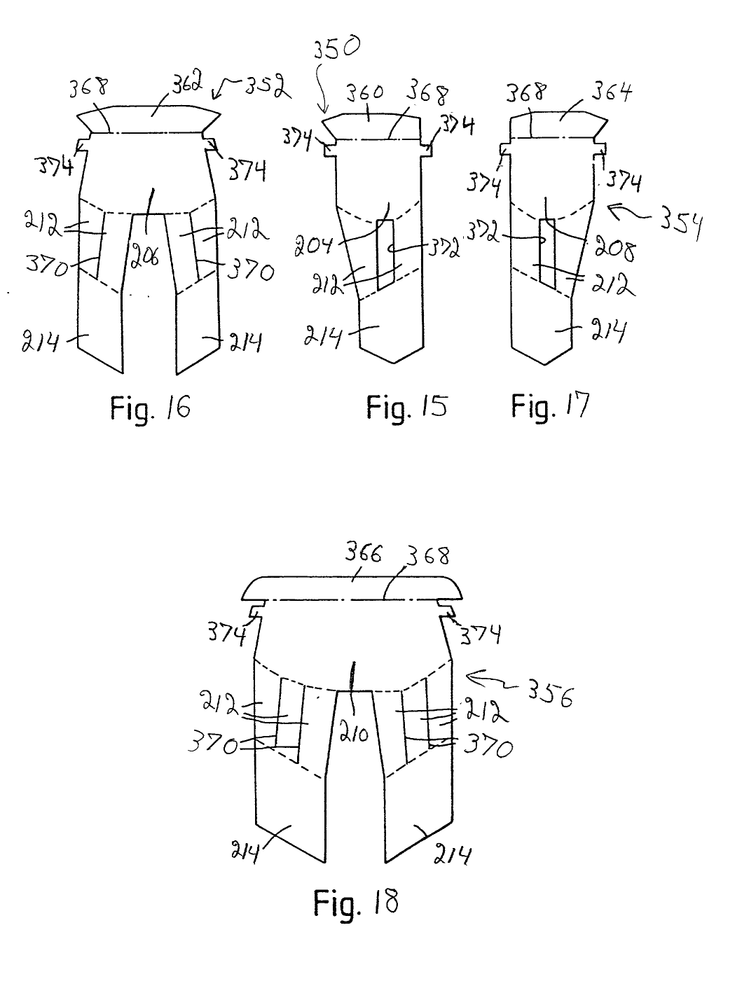

In the next step toward producing an aortic valve prosthesis, the trimmed tissue segment 306 is attached, such as by sewing, gluing or fusing, to a tissue sheet 310, which can be, for example, a sheet of bovine pericardium or other tissue. Tissue sheet 310 can be replaced with a fabric sheet or fabric tube formed from synthetic or natural fibers, which can be processed like tissue sheet 310. To assist with valve fabrication, slits 312 are cut, using a focused beam apparatus, in tissue sheet 310, as shown in FIG. 13. Slits 312 are formed without cutting tissue segment 306. After slits 312 are formed, tissue sheet 310 is manipulated to join the two opposite edges 314, 316 of tissue segment 306 to form a three leaflet valve structure. After the opposite edges are joined, the structure has a three dimensional shape requiring appropriate support to complete the cutting process. For example, the valve structure can be supported on a mandrel or the like connected to a stage.

The final trimming of the aortic valve involves trimming away excess portions of sheet 310 along the top and bottom of the formed valve. Referring to FIG. 14, the trimming along the top edge 309 is complete and one of the three sections of tissue sheet 310 has been trimmed away along the bottom of the valve. The other two sections of the tissue sheet 310 along the bottom of the valve are similarly trimmed. The trimming of excess tissue sheet 310 can be performed by using a three dimensional template after properly positioning the valve, including orientation of the leaflets, or using digital imaging to guide the cutting tool. The assembled valve is shown in FIG. 3.

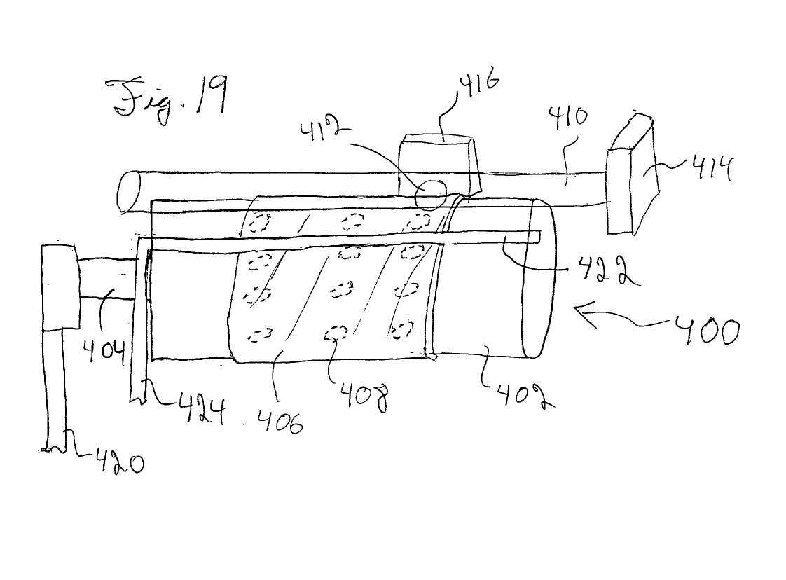

Four leaflet valve prosthesis 200 shown in FIG. 4 can be assembled from four leaflet sections that are joined together to form the valve. Referring to FIGS. 15-18, leaflet sections 350, 352, 354, 356 each have a section corresponding to one of leaflets 204, 206, 208, 210, respectively. Leaflet sections 350, 352, 354, 356 further include edge sections 360, 362, 364, 366, respectively. Edge sections 360, 362, 364, 366 together form edge 216 that is secured to the sewing ring by insertion between portions 218 and 220 of sewing ring 202, as shown in FIG. 4.

Referring to FIGS. 15-18, folds 368 separate edge sections 360, 362, 364, 366 from leaflets 204, 206, 208, 210. Specifically, leaflets 204, 206, 208, 210 are formed between folds 368 and chordae 212. Slits 370 are cut in leaflet sections 352, 356 to form chordae 212. Similarly, slots 372 are cut in leaflet sections 350, 354 to form chordae 212. Attachment sections 214 extend from the bottom of chordae 212. Additional structures, such as tabs 374, can be included to facilitate assembly of the prosthesis.

Leaflet sections 350, 352, 354, 356 are well suited to cutting using the template approach described herein. Tissue sheets, such as sheets of crosslinked bovine pericardium, can be used as the starting material. Since a sheet of tissue is cut to form the leaflet sections, there is no particular structure to be oriented using digital imaging. In preferred embodiments, the cutting beam follows a template, such as in FIG. 6, corresponding to the solid lines outlining the leaflet sections shown in FIGS. 15-18. The template can be a physical template or a virtual, i.e., digital, template. Very accurate cutting can be easily performed by directing the cutting beam toward the tissue sheet along the template corresponding to the preselected outline(s). The tissue sheet can be covered with a thin film of water to maintain the tissue in a hydrated state without significantly interfering with a laser or other beam cutting process. For example, a thin film of water can be maintained over the tissue by placing the tissue in a fluidized bed on a stage or by using a sprayer.

Following the cutting of the tissue, the tissue can be further processed, for example, by crosslinking the tissue. If desired, the tissue components can be placed in moist storage for an appropriate period of time prior to final assembly. When ready, the prosthesis can be assembled from the components. To assemble the tissue components, leaflet sections 350, 352, 354, 356 are attached to adjacent leaflet sections. Attachment sections 214 are secured into two groupings with one of the two attachment sections 214 of leaflet sections 352, 356 being attached to each group. Chordae 212 remain unattached to decrease interference with blood flow. Edge sections 360, 362, 364, 366 are attached to a sewing ring, as shown in FIG. 4. Assembly of a similar valve prosthesis is described U.S. Pat. No. 5,415,667 to Frater, entitled “Mitral Heart Valve Replacement,” incorporated herein by reference. Generally, a plurality of components can be cut from a single polymer sheet or tissue sheet, once the sheet is mounted on the platform.

Prostheses incorporating explants with intact leaflets from harvested valves generally are more amenable to cutting based on a digital image of the device due to their more complex structure. For example, the aortic valve of FIG. 1 and the mitral valve of FIG. 2 can be trimmed from a digital image to approximate a target image. For example, the aortic valve explant can be imaged after mounting the explant on a platform, such as with a mandrel, to determine the orientation and position of the leaflets. Then the top edge of the valve can be cut to form the desired scalloped shape from the target image without damaging the leaflets. After the top edge of the valve is formed, the bottom edge can be cut to form the desired structural relationship between the top edge and the bottom edge without damaging the leaflets. The cutting tool can be moved around the valve to obtain the appropriate cutting angle to make a particular cut without damaging intact structure.

Similarly, with the mitral valve of FIG. 2, annulus 152 is trimmed to a desired shape. Annulus 152 can be trimmed following the imaging of the top section of the valve. The cutting can then be performed without damaging the leaflets or the chordae. Similarly, the bottom portion of the valve can be imaged to cut papillary sections 160 to a desired size and shape while leaving the chordae intact. During the imaging and cutting with a focused beam, the valve explant can be supported on a suitable mount that does not interfere with the cutting. The tissue can be kept moist using an appropriate apparatus, as discussed above. Again, the cutting tool can be moved around the valve to obtain the appropriate cutting angle to make a particular cut without damaging intact structure.

As noted above, an imaging system can be used to measure tissue thickness with the tissue placed on a flat or curved surface with a known height. Imaging can be used to obtain an accurate thickness measurement. This can be performed with a flat support platform or by placing the material on a cylindrical mandrel, a conical mandrel or the like. Imaging along the surface of the mandrel can be used to identify variations in thickness to provide a guide for cutting to remove tissue outside the selected thickness range. A flat surface can be formed over a support surface. The support surface should be very flat, such as a polished granite surface. The granite surface and the flat platform serve as calibration reference points for the thickness measurement whether the tissue is on the flat surface or mandrel, such that the tissue can be cut as directed by the process control unit.

Referring to FIG. 19, thickness evaluating apparatus 400 includes a generally cylindrical mandrel 402 mounted on a rotating shaft 404 attached to platform 254 by arm 420. The mandrel can have different shapes and sizes, as desired. For example, the mandrel can be tapered for insertion into valves of different size. A tissue sheet 406 or valve explant is held on mandrel 402 using suction applied through holes 408, shown in phantom. Light beams 410, 412 can be projected along the mandrel (410) and/or perpendicular to the mandrel axis (412) or in other convenient directions to measure the thickness. For example, thickness can be correlated with the amount of light detected with detectors 414, 416, which can be photomultiplier tubes or diode detectors, since thicker tissue will block more of the light.

To keep the tissue moist while mounted on mandrel 402, a sprayer 422 can be used. Sprayer 422 can be connected to a water, buffer or other suitable liquid source through arm 424.

Once the thickness is evaluated, the tissue can be cut to remove portions that do not have a thickness within a desired range. Cutting can be performed with the tissue still mounted to the mandrel, for example, by rotating the mandrel with shaft 404 to cut the tissue with a focused beam. For example, tissue with a thickness outside of the range from about 2 mm to about 2.5 mm can be removed from a tissue segment. This mandrel can also be used for other imaging and focused beam cutting embodiments described above.

Completion of the Medical Device

The medical devices of interest include tissue, as described above. In many embodiments, multiple tissue elements are assembled and/or additional non-tissue components are attached to the device to complete the medical device. In particular, the medical devices can also include other biocompatible materials, such as polymers, ceramics and metals, along with the tissue.

Tissue cutting can be performed before or after attachment of additional tissue elements and non-tissue elements, depending on the design of the device. In particular, it may be desirable to cut additional tissue elements and/or non-tissue components along with or simultaneously with a particular tissue element. For these embodiments, the tissue is attached to the other components prior to cutting. Alternatively, additional components may interfere with the tissue cutting if they are attached to the tissue prior to cutting. For these embodiments, the additional components are attached after the tissue element is cut.

Appropriate ceramics for incorporation into a prosthesis include, without limitation, hydroxyapatite, alumina and pyrolytic carbon. Biocompatible metals include, for example, titanium, cobalt, stainless steel, nickel, iron alloys, cobalt alloys, such as Elgiloy®, a cobalt-chromium-nickel alloy, MP35N, a nickel-cobaltchromium-molybdenum alloy, and Nitinol®, a nickel-titanium alloy.

Polymeric materials for prostheses can be fabricated from synthetic polymers as well as purified biological polymers. These polymers can be combined with tissue in forming the medical device, or they can be used alone or with other nontissue materials. Appropriate synthetic materials include hydrogels and other synthetic materials that cannot withstand severe dehydration. Suitable polymers include bioresorbable polymers that are gradually resorbed after implantation within a patient. In addition, the polymers can be coated, for example, with other polymers, biological agents, such as growth factors, inert materials, such as carbons, and/or metals, to induce desired properties to the polymers.

Appropriate synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers. These synthetic polymeric materials can be formed into fibers or yarns and then can be woven or knitted into a mesh of fabric to form a matrix or substrate. Alternatively, the synthetic polymer materials can be molded or cast into appropriate forms.

Biological polymers can be naturally occurring or produced in vitro by fermentation and the like or by recombinant genetic engineering. Suitable biological polymers include, without limitation, collagen, elastin, silk, keratin, gelatin, polyamino acids, polysaccharides (e.g., cellulose and starch) and copolymers thereof.

As an example, the heart valve prosthesis 100 in FIG. 1 has a fabric cover 102 on the outside of the valve. This fabric preferably is formed from synthetic polymers, such as polyester. Generally, the cover would be attached after the tissue is cut since the suture lines follow the cut, although the fabric could be cut along with the tissue and sutured following the cutting. Although the discussion focuses on tissue containing prostheses, relevant medical devices can also be formed with just non-tissue materials.

Storage, Distribution and Use

Following cutting, the tissue, possibly formed into a prosthesis, can be stored. Preferred storage techniques minimize the risk of microbial contamination. For example, the tissue can be stored in a sealed container with sterile buffer, saline solution and/or an antimicrobial agent, such as glutaraldehyde or alcohol.

For distribution, the bioprostheses generally are placed in sealed and sterile containers. To ensure maintenance of acceptable levels of sterility, the tissue can be transferred to the sterile container using accepted aseptic protocols. The containers can be dated such that the date reflects the maximum advisable storage time.

The containers generally are packaged with instructions for the use of the medical devices along with desired and/or required labels. The containers are distributed to health care professionals for surgical implantation of the prostheses. The implantation is performed by a qualified health care professional. The surgical implantation generally involves the replacement or supplementation of damaged tissue with the prosthesis.

The embodiments above are intended to be illustrative and not limiting. Additional embodiments are within the claims. Although the present invention has been described with reference to preferred embodiments, workers skilled in the art will recognize that changes may be made in form and detail without departing from the spirit and scope of the invention.

What is claimed is:

1. A method for producing a prosthesis, the method comprising at least partially cutting a material segment with a beam wherein the cutting is controlled by a process control unit to cut the material to correspond to a target image.

2. The method of claim 1 wherein the pattern is determined according to a preselected template.

3. The method of claim 1 wherein the material segment comprises a tissue segment separated from an organism.

4. The method of claim 3 wherein the tissue segment comprises a tissue sheet.

5. The method of claim 1 wherein the material segment comprises a polymer.

6. The method of claim 1 wherein the target image has a leaflet section.

7. The method of claim 1 wherein the target image is determined by
a) forming a digital image of the material segment;
b) comparing the digital image to a target image to evaluate the difference between the digital image and the target image; and
c) determining a cutting pattern based on the difference.

8. The method of claim 7 wherein the digital image is formed using a video camera.

9. The method of claim 7 wherein the digital image is formed by a scanning phase measurement.

10. The method of claim 7 wherein determining the cutting pattern involves forming the cutting pattern based on the border between the digital image and the target image.

11. The method of claim 7 wherein the cutting pattern is selected to avoid cutting any material that forms a portion of the target object.

12. The method of claim 7 further comprising orienting the digital image relative to the target image prior to comparing the digital image with the target image.

13. The method of claim 1 wherein the beam comprises a laser beam, and wherein the laser beam passes through frequency shifting optics.

14. The method of claim 1 wherein the beam comprises infrared laser light.

15. The method of claim 1 wherein the beam comprises visible laser light.

16. The method of claim 1 wherein the beam comprises ultraviolet laser light.

17. The method of claim 1 wherein the beam comprises a fluid.

18. The method of claim 1 wherein the cutting comprises moving beam directing optics to an appropriate location to direct the beam to the selected position on the material segment.

19. The method of claim 1 wherein the cutting comprises moving a support platform supporting the material to direct the light to a selected portion of the tissue.

20. The method of claim 1 wherein the at least partially cutting comprises partially cutting the material segment.

21. The method of claim 1 wherein the at least partially cutting comprises complete cutting of the material segment.

22. The method of claim 1 wherein the process control unit comprises a digital processor.

23. The method of claim 1 further comprising assembling cut components to form a prosthesis.

24. An apparatus for cutting a tissue segment, the apparatus comprising:
a) a tissue segment;
b) a support platform supporting the tissue segment;
c) a beam generator oriented to direct a beam at the tissue segment; and
d) a process control unit that controls the relative position of the support platform and the beam.

25. The apparatus of claim 24 further comprising a motor that changes the relative position of the support platform and the beam.

26. The apparatus of claim 25 wherein the process control unit actuates the motor to adjust the relative position of the support platform and the beam.

27. The apparatus of claim 25 wherein the motor is operably connected to the support platform to move the support platform relative to the beam.

28. The apparatus of claim 25 wherein the process control unit comprises a digital processor operably connected to the motor, wherein the processor controls the motor based on a target image.

29. The apparatus of claim 25 wherein the process control unit comprises a manual control that controls the actuation of the motor.

30. The apparatus of claim 24 wherein the support platform comprises a flat surface contacting the tissue.

31. The apparatus of claim 24 wherein the support platform comprises a mandrel supporting the tissue segment.

32. The apparatus of claim 31 wherein the mandrel is cylindrical or tapered.

33. The apparatus of claim 24 further comprising optical elements within the beam generated by the beam source to redirect the beam, and wherein the process control unit is operably connected to the optical components to move the optical components relative to the support platform.

34. The apparatus of claim 24 wherein the support platform comprises a fluidized bed.

35. The apparatus of claim 24 wherein the support platform comprises a vacuum fixture.

36. The apparatus of claim 24 further comprising a imaging device comprising a detector, wherein the imaging device is connected to the process control unit to form a digital image of the tissue segment.

37. The apparatus of claim 36 wherein the imaging device comprises a digital video camera.

38. The apparatus of claim 24 wherein the tissue segment comprises a heart valve explant with intact leaflets.

39. The apparatus of claim 24 wherein the tissue segment comprises a tissue sheet.

40. The apparatus of claim 39 wherein the tissue sheet has a film of water covering the tissue.

41. The apparatus of claim 24 wherein the beam source comprises a laser.

42. The apparatus of claim 24 further comprising beam directing optics mounted on a motorized stand, such that movement of the motorized stand alters the position of the beam on the tissue segment.

43. The apparatus of claim 24 wherein the beam source comprises a fluid jet.

44. The apparatus of claim 24 further comprising a humidity control to maintain high humidity in the environment surrounding the tissue.

45. A heart valve prosthesis comprising a tissue segment separated from the host, the tissue having a cauterized edge.

46. A method of cutting a tissue sheet to remove portions of the tissue sheet having different thicknesses, the method comprising:
imaging the tissue sheet on a smooth surface to evaluate the thickness of the tissue sheet at different points; and
cutting the tissue sheet to separate portions of the tissue sheet with a thickness outside of a selected range.

47. The method of claim 46 wherein the tissue sheet is mounted on a flat support platform that serves as a calibration reference point.

48. The method of claim 46 wherein the tissue sheet is mounted on a mandrel.

49. The method of claim 46 wherein the imaging is performed with a laser and a detector.
